(19) United States
(12) Patent Application Publication
(10) Pub. No.: US 2002/0121280 A1
(43) Pub. Date: Sep. 5, 2002

(54) Method for myocardial revascularization

(75) Inventor: Lucas S. Gordon, Redmond, WA (US)

Correspondence Address: LYON & LYON LLP, 633 WEST FIFTH STREET, SUITE 4700, LOS ANGELES, CA, 90071, US

(73) Assignee: PharmaSpec Corporation

(21) Appl. No.: 09/798,599

(22) Filed: Mar. 1, 2001

(51) Int. Cl. A61M 31/00; A61B 19/00

(52) U.S. Cl. 128/898; 604/507

(57) ABSTRACT

A method is disclosed for revascularizing ischemic tissue by creating a continuous channel from a non-ischemic to an ischemic area of the myocardium. A penetrating tip at the distal end of a catheter is advanced through specific areas of the myocardium. Healing of the channel results in angiogenesis of new blood vessels along its path, thus establishing a continuous vascular connection between the vascular beds in the non-ischemic myocardium and in the ischemic myocardium. The channel may originate from a blood vessel on the heart or from the inner or outer surface thereof. Preferably, the channel comprises a split path, formed by separating a thin layer of myocardium rather than creating a hole therethrough. Growth of the new blood vessels is further enhanced by placing an angiogenic growth factor in at least a portion of the channel.

### RELATED APPLICATION DATA

This application claims priority on U.S. Provisional Patent Application S/N [not-yet-assigned; Lyon & Lyon attorney docket 259/237], filed Dec. 12, 2000, the disclosure of which is fully incorporated herein by reference.

### FIELD OF THE INVENTION

The invention relates to methods and medical apparati used to revascularize ischemic tissue. In particular, the invention relates to the revascularization of ischemic myocardial tissue.

### BACKGROUND

A number of different diseases can affect the human heart. Coronary artery disease, for example, is caused by a buildup of plaque in coronary arteries. This can result in the blockage of blood flow within one or more arteries of the heart, which may lead to ischemia (i.e., lack of oxygen) in tissues normally supplied with blood through the blocked arteries. Ischemic tissue is tissue at a low oxygen state, usually due to an obstruction of the arterial blood supply or inadequate blood flow that leads to hypoxia in the tissue; non-ischemic tissue is tissue at a normal oxygen state. Coronary arteries seem to be particularly vulnerable both to gradual plaque buildup and to a catastrophic heart attack when a plaque ruptures and triggers a total thrombotic occlusion of the artery. If the occlusion remains for a period of more than about six hours, myocardium (heart muscle) that is devoid of oxygenated blood flow will die.

Discrete plaque formations may be effectively treated by percutaneous transluminal coronary angioplasty (PTCA). In PTCA, a catheter incorporating an inflatable balloon at the distal end is inserted into the patient's femoral artery and advanced up the aorta and into the diseased coronary artery. After positioning the balloon within the obstruction, the balloon is inflated, compressing the plaque and stretching the artery wall. The action of the balloon results in an enlargement in the restricted area, or stenosis, providing increased blood flow to the area of myocardium fed by that artery. Plaque may also form in a diffuse manner within a vessel and is much more difficult to treat with PTCA. A gradual build up of plaque in an artery, whether diffuse or discrete, reduces the flow of blood and prevents the myocardium in the affected area from contracting. Often the muscle tissue is still alive if a minimal quantity of blood flow is maintained. This type of ischemic myocardial tissue is characterized as dormant, wherein, if blood flow to the tissue is re-established, a portion or all of its contractile function may be restored.

One treatment that has been attempted to restore blood flow to dormant myocardial tissue is percutaneous myocardial revascularization (PMR). In one version of PMR, a catheter incorporating an optical fiber is threaded from the femoral artery of the patient into a chamber of the heart, such as the left ventricle. The tip of the fiber is placed against the inner surface of the ventricle in a location of the affected myocardium. A high power laser is pulsed through the fiber, ablating a portion of myocardial tissue and forming a discrete channel approximately one millimeter in diameter and several millimeters deep in the myocardium. Generally, channels formed by this process run perpendicular to the inner surface of the ventricle. The process is repeated in a square grid pattern of about one centimeter in the area of targeted ischemic tissue. An example of the resulting channel formation is illustrated in FIG. 1.

Originally, PMR was thought to perfusion direct perfusion of the myocardial tissue with oxygenated blood from a chamber of the heart. Many patients treated in this manner reported a decrease in pain upon exertion. Further investigation, however, has shown that the channels actually clot off and heal with formation of scar tissue, resulting in no direct blood flow through the channels. Interestingly, localized new arteries and arterioles were seen in close proximity encircling each channel. From these observations it was then theorized that these new blood vessels, formed by a process during wound healing called angiogenesis, were the actual source of patient improvement. In angiogenesis, tissue revascularizes by developing new blood vessels near the site of an injury. Tissue injury that stimulates angiogenesis can be caused by any type of damage to the tissue in which energy is applied to it by means including mechanical, electromagnetic, thermal (hot or cold), chemical, nuclear radiation, and biological. It is now believed that macrophages at the surface of a wound express angiogenic growth factors that promote revascularization in wound healing. An angiogenic growth factor is a substance that causes proliferation of new blood vessels by angiogenesis.

A promising new genre of therapeutic agents are termed “gene therapy” agents. The therapeutic agents supplement the normal angiogenic growth factors that are present during normal wound healing. Gene therapy is a technique where DNA is inserted directly into cells to replace, alter, or supplement a gene within the cell. One such gene therapy promotes angiogenesis in an affected heart muscle. The therapy may simply comprise delivering the angiogenic protein, such as vascular endothelial growth factor (VEGF). Alternately, placement of genetic material that manufactures the protein in-situ may be used—for example, the use of viral vectors to transfer genetic material to target cells. This method involves the use of incorporating heterologous DNA sequences into the genome of the virus. Additionally, both RNA and DNA recombinant viruses have been employed.

Recently, a placebo-controlled clinical study of PMR showed equal or superior results in patients who were treated with the sham procedure compared with patients who actually received PMR. This indicates that even though new blood vessels were formed, they were ineffective in improving perfusion to the ischemic tissue; thus, the placebo effect was the primary factor for the apparent improvement. It is theorized that the failure of PMR to produce results better than the sham procedure is because the new vessels that are formed within the ischemic tissue are not supplied with oxygenated blood from outside the ischemic tissue. Many variations of laser PMR have been proposed, however, all rely on the placement of channels in grid patterns within the ischemic area. For example, U.S. Pat. No. 6,053,911 to Ryan et al., the contents of which are fully incorporated herein by reference, teaches a method of transvascular transmyocardial revascularization (TMR). In this method, a channel originates from a coronary blood vessel and enters the myocardium, traversing it in a direction substantially parallel to the walls of the heart. Because these channels must originate from coronary blood vessels, they are limited as to their positioning and orientation.

Despite the existing techniques for myocardial revascularization, there remains a need for an effective method and apparatus that will take advantage of the phenomenon of angiogenesis to provide actual and useful revascularization of ischemic myocardial tissue.

### SUMMARY OF THE INVENTION

Methods and apparatus are disclosed for revascularizing ischemic tissue by creating a continuous channel from an area of the myocardium having an adequate arterial blood supply (non-ischemic) to an area that has inadequate blood supply (ischemic). A resulting network of new blood vessels are formed within the affected myocardium that are adequately supplied with blood from healthy myocardial tissue. In this manner, stimulating angiogenesis is provided in the needed locations, while minimizing or eliminating the distribution of angiogenic growth factors to undesirable areas of the body.

One method for delivery of angiogenic growth factors according to one aspect of the invention includes advancing a catheter through a patient's vasculature to a coronary artery or coronary vein. A penetrating tip at the distal end of the catheter is pushed through the vessel wall and then advanced into specific areas of the myocardium. In particular, the tip is advanced through both normal myocardial tissue and adjacent ischemic myocardial tissue, creating a continuous channel between the two. The order of passing through normal and ischemic myocardium is not significant, but the path must cross both types of tissue to establish a continuous path of new vascular vessels between the vascular beds in the non-ischemic and ischemic myocardial tissue, thereby supplying the non-ischemic tissue with blood. The catheter path preferably runs parallel to the inner and outer surfaces of the heart chamber, or at a very shallow angle relative to the chamber wall. In the latter case, the catheter path may continue for a substantial distance without encountering either the inner or outer surface of the heart chamber. Healing of the channel results in angiogenesis of new blood vessels along the entire path, thus establishing a continuous vascular connection between the vascular beds in the non-ischemic myocardium and in the ischemic myocardium.

In another embodiment, a channel is formed from the inner surface of the heart chamber and penetrating into the myocardium, passing continuously through both ischemic and non-ischemic tissue in the myocardium. Alternatively, a similar channel may be formed from the outer surface of the heart, although that approach requires surgical access through the pericardium and may be less desirable.

In accordance with another aspect of the invention, the path created by the advancing catheter is a hole formed by removing or ablating tissue with a channeling catheter. If a discrete hole is desired, a number of energy sources, such as laser, radio frequency, or mechanical ablation techniques, e.g., ultrasonic or lower frequency vibrations or rotations, are suitable for removing tissue. Preferably, the channel comprises a split path formed by a catheter tip that merely separates or dissects the myocardial tissue substantially in a plane as the tip is advanced, removing little or no tissue. A split path, as compared with a hole, results in less undesirable scar tissue and may have a greater cross sectional surface area, which results in more revascularization.

In accordance with another aspect of the invention, the growth of the new blood vessels may be further enhanced by placing an angiogenic growth factor in at least a portion of the channel. A channel may be formed long, narrow, and blind, i.e., without an outlet. Angiogenic growth factors deposited such a channel are unlikely to be expressed or washed out, even with the compressing action of the beating heart. Accordingly, growth factors are prevented from reaching other parts of the patient.

In accordance with yet another aspect of the invention, diagnostic agents may be deposited in the channel for verifying the correct location of the path. Suitable diagnostic agents include ionic and non-ionic fluoroscopic contrast agents in conjunction with x-ray equipment.

Other aspects and features of the present invention will become apparent from consideration of the following description taken in conjunction with the accompanying drawings.

### BRIEF DESCRIPTION OF THE DRAWINGS

Preferred embodiments are illustrated by way of example, and not by way of limitation, in the figures of the accompanying drawings, in which like reference numerals refer to like components, and in which:

FIG. 1 is an anterior view of the heart in partial cross section, showing prior art PMR channels within the wall of the heart;

FIG. 2 is an anterior view of the heart, showing a myocardial revascularizing catheter in a coronary artery;

FIG. 3 is a cross section of the wall of the heart, showing the distal end of a myocardial revascularization catheter in an artery and a continuous path of angiogenesis;

FIG. 4 is an anterior view of the heart in partial cross section, showing a continuous path of angiogenesis within the left ventricle;

FIG. 5 is a cross section of the wall of the heart, showing a continuous path of angiogenesis starting at a location within the left ventricle;

FIG. 6 is a side view of the distal end of the myocardial revascularizing catheter;

FIG. 7 is a cross section of the distal end of the myocardial revascularizing catheter;

FIG. 8 is a cross section side view of the distal end of the myocardial revascularizing catheter;

FIG. 9 is an anterior view of the heart in partial cross section, showing the distal end of a myocardial revascularization catheter in the left ventricle; and

FIG. 10 is a view of the wall of the heart, showing the distal end of the myocardial revascularizing catheter creating a path within the left ventricle.

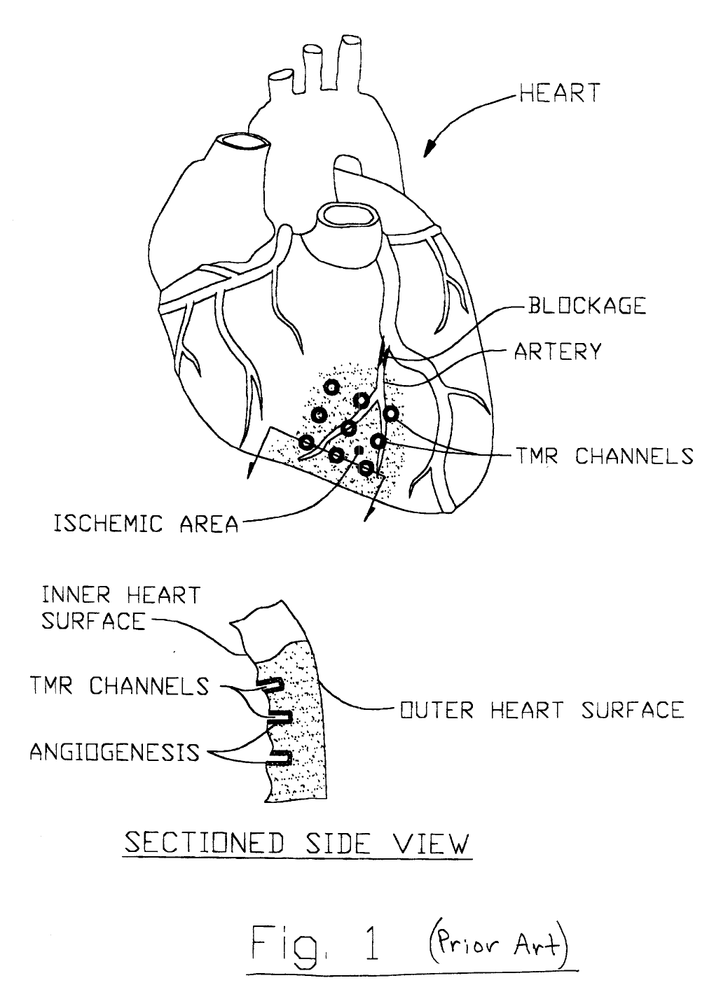

### DETAILED DESCRIPTION OF THE PREFERRED EMBODIMENTS

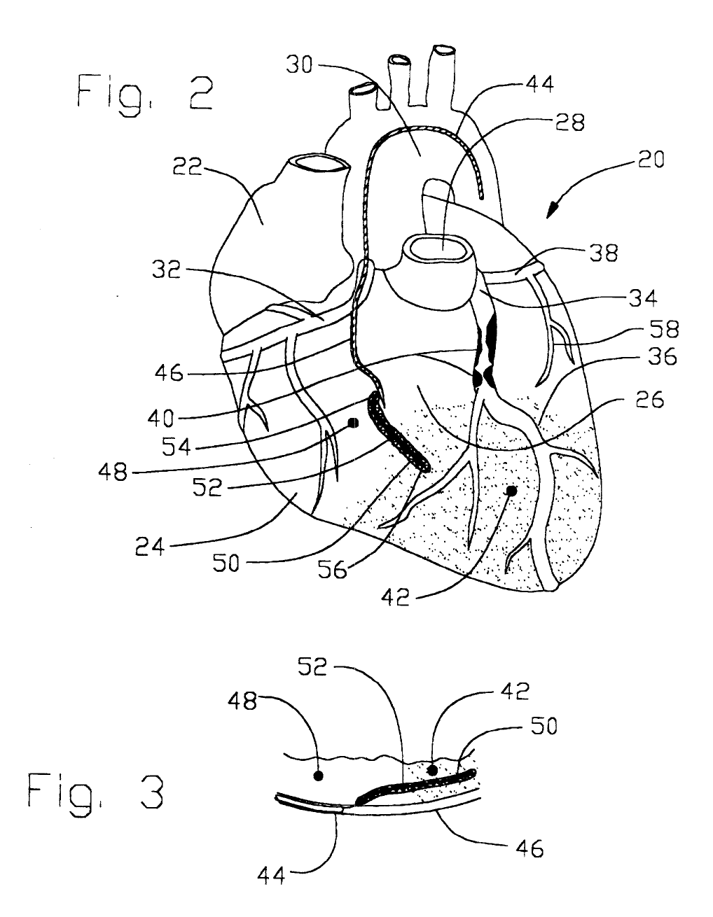

An anterior view of the human heart 20 is shown in FIG. 2. It includes the right atrium 22, the right ventricle 24, the left ventricle 26, the pulmonary artery 28, and the aortic arch 30. A coronary artery is a blood vessel that supplies the heart tissues with oxygenated blood, and a coronary vein is a blood vessel that removes deoxygenated blood from the heart tissues. Originating at the base of the aorta is the right coronary artery (RCA) 32 and the left coronary artery (LCA) 34. The LCA 34 divides into the left anterior descending artery (LAD) 36 and the circumflex 38 artery. A stenosis 40 is shown in the LAD 36, resulting in the shaded ischemic area 42 in the lower portion of the left ventricle 26.

The distal portion of a myocardial revascularization catheter 44 is shown extending over the aortic arch 30, into the RCA 32, and down the first diagonal artery branch 46. The catheter 44 forms a continuous path (or channel) 52 from a point 54 located in non-ischemic myocardial tissue 48 to a point 56 located in ischemic myocardial tissue (shaded) 42. The healing of tissue along the path 52 produces a corresponding continuous revascularization path 50 by angiogenesis.

Additional revascularization paths may be generated from the same diagonal artery branch 46 or from another artery 58 that lies within the non-ischemic area. Similarly, the coronary veins generally lie next to the arteries, and similar revascularization paths may be generated from any of the veins that lie within the non-ischemic area. Alternatively, paths may be formed by advancing the catheter down an artery or vein to a point within an ischemic area and directed to a non-ischemic area. Whether the path begins or ends in an ischemic area is not material, as long as there results in a continuous path from ischemic to non-ischemic myocardial tissue.

In one embodiment, a diagnostic agent is injected into the channel 52 to verify the location of the path within the myocardium. A diagnostic agent is any chemical or other material that is used to determine the cause of illness or to provide any visual display of structural or functional patterns of organs, tissues or of a given therapy. It also includes physiologic and metabolic responses to physical or chemical stimuli or to a given therapy. Examples of suitable diagnostic agents include radiographic, ultrasonic, or magnetic resonance visualizing agents; fluorescing dyes responsive to temperature, pH, oxygen, CO₂, sodium, potassium, or calcium levels; and the like. A preferred diagnostic agent is a radiopaque contrast imaging solution, which is capable of being visualized with a fluoroscope.

With regard to the catheter 44, radiopacity may be created by, e.g., impregnating the catheter with a radiopaque substance, impregnating the outer surface of the catheter with a radiopaque substance, coating the carrier with a radiopaque substance, or attaching a radiopaque component to the catheter. A carrier is any fluid or solid vehicle material used to transport an agent, and suitable carrier materials include aqueous fluids, solvents, detergents, lipids, biological cells, electrolytes, nutrients, oncotic agents, buffers, alcohols, thixotropics, stimuli-sensitive polymers (liquid to gel), phase changeable solid polymers (liquid to solid), hydrogels, and the like.

In another embodiment, an angiogenic growth factor is deposited within the channel 52 to stimulate additional angiogenesis and increase the rate of growth. Many angiogenic growth factors occur naturally within the body, for example, fibroblast growth factors (FGF) and vascular endothelial growth factors (VEGF). Synthetic forms or equivalents are also suitable, as are various organisms that express such factors such as viral vectors. Autologous endothelial cells are also thought to be helpful in promoting angiogenesis and may be added to the channel 52 in combination with other angiogenic growth factors. Other angiogenic growth factors that generate sub lethal tissue damage may also stimulate angiogenesis without creating scar tissue. Typical substances suitable for this purpose include acids or bases that cause chemical injury and substances that cause a thermal injury, thus stimulating increased angiogenesis.

FIG. 3 shows a cross section of the wall of the heart in FIG. 2 showing the continuous path 52 extending from the wall of artery 46 through non-ischemic myocardial tissue 48 to ischemic tissue 42. Alternatively, the path 52 could be made to extend continuously from the artery 46 through ischemic myocardial tissue 42 to non-ischemic tissue 48.

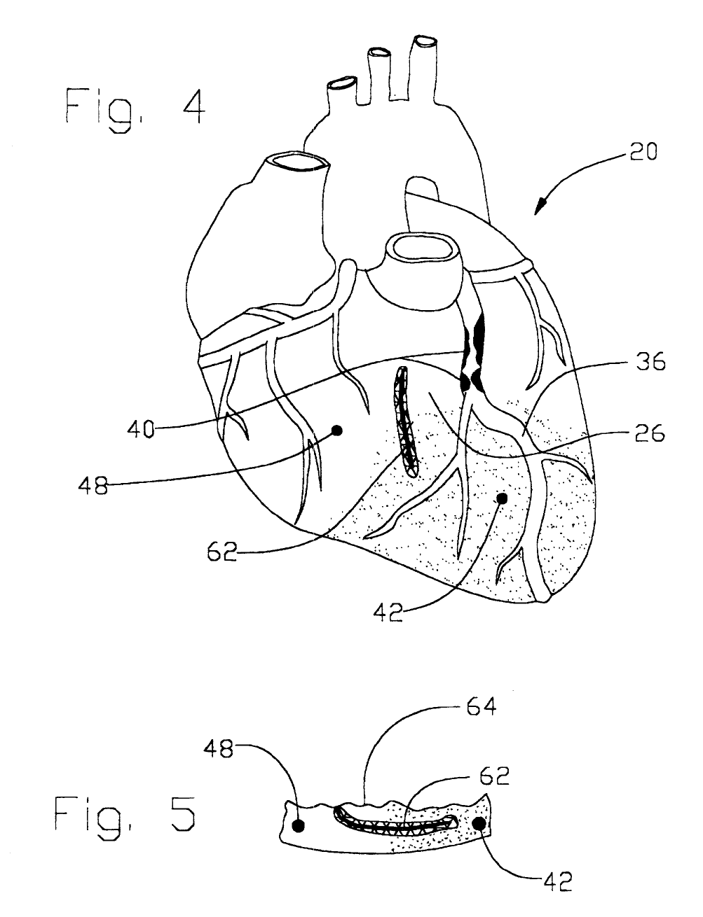

FIG. 4 depicts another anterior view of a human heart 20, showing a revascularization path 62 from within the wall of the left ventricle. FIG. 5 is a cross section of the wall of the left ventricle of the heart showing the continuous revascularization path 62 between non-ischemic myocardial tissue 48 and ischemic tissue (shaded) 42. In this illustration, the continuous revascularization path 62 has been formed starting from the inside surface (endocardium) 64 of the left ventricle by a catheter percutaneously placed within the left ventricle.

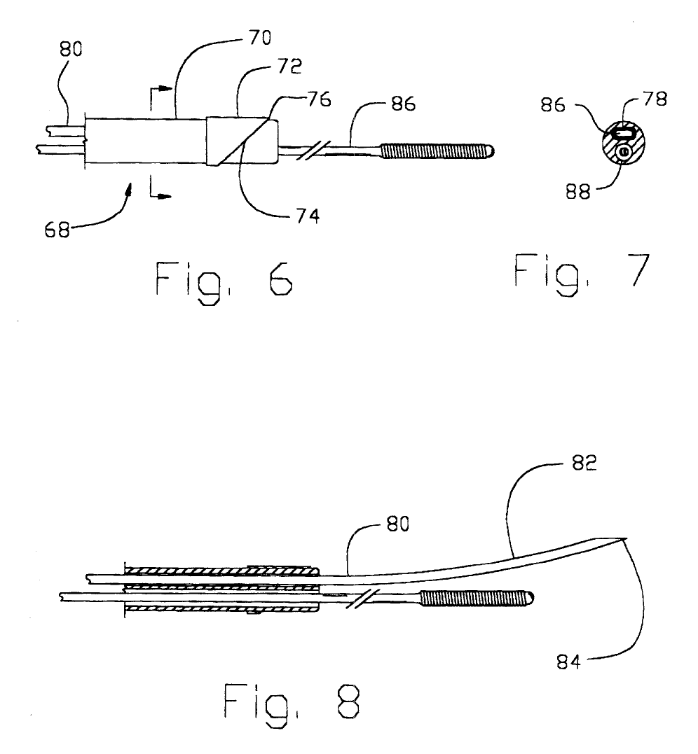

FIGS. 6, 7, and 8 show the distal portion 68 of a catheter for creating a continuous path in myocardial tissue as illustrated in the previous figures. The catheter includes a shaft 70 located within which are two lumens 78,88. A radiopaque band 72 is attached to the shaft 70. The catheter shaft 70 may be constructed of a flexible polymer, such as polyamide, polyethylene, polypropylene, polyurethane, poly vinyl chloride, or polyether block amides. The radiopaque band 72 is preferably made of a suitably high density material so that it can be readily detected, such as tungsten, platinum or gold. Radiopacity of lower density materials may be increased by coating them with high density materials, such as platinum or gold, by processes known in the art like plating or ion implantation. Radiopaque band 72 has a distal face 74 formed at an oblique angle to the axis of the catheter shaft 70, which creates a point 76 that rotationally aligns with oval lumen 78 on the cross section of the catheter shaft 70.

Within oval lumen 78 is an oval wire 80 slidably disposed in the lumen 78. Wire 80 may be made of a variety of suitable materials, including metals such as tungsten, stainless steel, or nickel-titanium alloys, or polymers such as polyamide, polyimide, or polycarbonate. Wire 80 extends from the distal end of the catheter through the entire catheter and out of the proximal end (not shown). Wire 80 is formed with a curve bias at its distal end 82, wherein the curvature of the wire 80 approximately matches the curvature of the wall of a typical human heart. The oval shape of catheter lumen 78 and slidable wire 80 insures that wire curve 82 aligns properly with radiopaque marker point 76, thereby allowing a user of the catheter to properly align it. Wire 80 has a point 84 formed at the distal end to easily penetrate the myocardial tissue, and wire 80 also contains therewithin a lumen 86, which extends the full length of the wire.

The point 84 is preferably of a standard non-coring design, such as those found on standard hypodermic needles, to prevent tissue from entering and thus occluding the lumen 86. Lumen 86 is further adapted for depositing a therapeutic agent within the path formed through myocardial tissue by the slidable wire 80.

The myocardial tissue that is removed is replaced by non-functional scar tissue in the voids where the tissue was removed. It is thus desirable to minimize the replacement of functional myocardial tissue with scar tissue, whether in ischemic or non-ischemic areas of the myocardium. Accordingly, in another aspect of a preferred embodiment, wire 80 is adapted to cut a split path through the myocardial tissue when advanced therethrough. The wire 80 adapted to cut a split path is one that merely separates or dissects the myocardial tissue substantially in a plane, removing little or no tissue. Typically, the split path collapses after the catheter is removed. A split path, as compared with a hole, more closely resembles a blade's cut than a bored hole. Accordingly, a split path channel generally results in less undesirable scar tissue and greater cross sectional surface area, which results in more revascularization.

Generally, a therapeutic agent is any chemical or other material that is used in the treatment of a disease or disorder. Examples of therapeutic agents are gene therapy agents, biological cells, tissues or precursors, antibiotics, antineoplastics, enzymes, vitamins, hormones, antivirals, radiation (via radiation sources such as cobalt, radium, and radioactive sodium iodide), anticoagulants, hemostatic agents, hepatoprotectants, vasodilators, acids, bases, inflammatory agents, and the like. Further, any therapeutic agent that can be adhered to the surface of a carrier material or impregnated into the carrier material may be administered using the devices and methods disclosed herein by administering the carrier material.

A method for myocardial revascularization according to one embodiment of the invention comprises first creating an access site in the femoral artery. Such a technique is standard in the arts of, e.g., performing angioplasties and other similar procedures. Using standard techniques, the catheter shaft 70 is advanced through the arterial system until radiopaque marker 70 is located within an artery overlying an ischemic 42 or non-ischemic 48 area of the myocardium. If placement of the catheter tip is to be made in a coronary vein, the access site would be the femoral vein, the jugular vein, or another easily accessible vein large enough to accommodate the catheter.

The catheter 68 is then rotated until the radiopaque marker point 76 is aligned so that the point 76 lies toward the myocardium. The proximal end of slidable wire 80 is then advanced to extend from the proximal end of the catheter, thereby puncturing the wall of the coronary vessel and extending into the myocardium. This action creates a continuous channel 52 that passes through ischemic 42 and non-ischemic 48 areas of the myocardium.

Also formed within catheter shaft 70 is a lumen 88, with guide wire 86 slidably inserted therein. Guide wire 86 may be of any standard type known in the art. Lumen 88 may extend through the entire length of shaft 70 to the proximal end in a standard “over the wire” style of catheter. In an alternate preferred embodiment, lumen 88 may extend about 25 centimeters proximally, before breaking out through the outer surface of catheter shaft 70 in a standard “rapid exchange” style of catheter, also well known in the art.

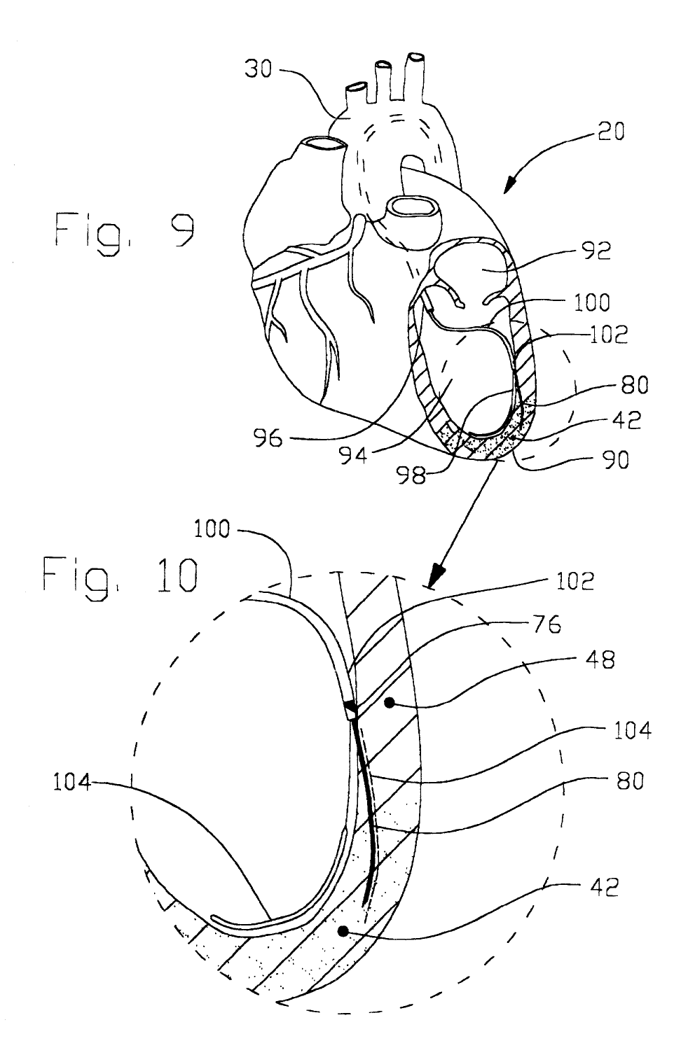

Referring to FIGS. 9 and 10, an anterior view of the human heart 20 is shown. A section of the heart has been cut away to show the left atrium chamber 92 and the left ventricle chamber 94. The heart comprises an area of ischemic myocardial tissue (shaded) 42 in the apex 90 of the left ventricle chamber 94. The ischemic tissue could have been caused by, e.g., a blockage in an artery that deprived the tissue of oxygenated blood flow. A guide catheter 96 and guide wire 98 have been advanced over the aortic arch 30 and into the left ventricle chamber 94. Guide wire 98 preferably has a pre-curved end 104 to match the shape of the inner surface of the left ventricle chamber. Catheter 100 is advanced over guide wire 98 and within guide catheter 96 until its distal end 102 is at a desired location on the ventricle wall next to a non-ischemic portion 48 of the myocardium. The catheter 100 is rotated until the radiopaque marker point 76 is aligned with the heart so that the point 76 lies toward the myocardium 48. Advancing slidable wire 80 distally punctures the wall of the left ventricle 94 and creates a continuous path 104 through non-ischemic myocardial tissue 48 and into ischemic myocardial tissue 42. Slidable wire 80 may also contain a lumen 88 and a non-coring point 84 adapted to administer a therapeutic or diagnostic agent into the continuous path 104.

While preferred embodiments and applications have been shown and described, as can be appreciated by those of ordinary skill in the art, the invention can be embodied in other specific forms without departing from the inventive concepts contained herein. The presently disclosed embodiments, therefore, should be considered as illustrative, not restrictive. Accordingly, the invention should not be limited except by the scope of the appended claims and their equivalents.

What is claimed is:

1. A method for revascularizing myocardial tissue comprising ischemic and non-ischemic areas, comprising:
creating a channel in the myocardial tissue of the heart extending continuously from an ischemic area of the myocardial tissue to a non-ischemic area of the myocardial tissue.

2. The method of claim 1, wherein the channel extends continuously from a coronary blood vessel into areas of both ischemic and non-ischemic myocardial tissue.

3. The method of claim 1, wherein the channel extends continuously from an inner surface of the heart into areas of both ischemic and non-ischemic myocardial tissue.

4. The method of claim 1, wherein the channel extends continuously from an outer surface of the heart into areas of both ischemic and non-ischemic myocardial tissue.

5. The method of claim 1, further comprising injecting a therapeutic agent into at least a portion of the channel.

6. The method of claim 1, wherein the resultant channel is substantially a split path.

7. The method of claim 1, wherein the step of creating a channel is repeated to produce a plurality of channels in the myocardium, each channel extending continuously from an area of ischemic myocardial tissue to an area of non-ischemic myocardial tissue.
